(19) United States
(12) Patent Application Publication
(10) Pub. No.: US 2005/0191636 A1
(43) Pub. Date: Sep. 1, 2005

(54) Detection of STRP, such as fragile X syndrome

(75) Inventor: Soonkap Hahn, San Clemente, CA (US)

Correspondence Address: FITCH EVEN TABIN AND FLANNERY, 120 SOUTH LA SALLE STREET, SUITE 1600, CHICAGO, IL, 60603-3406, US

(73) Assignee: Biocept, Inc., Carlsbad, CA (US)

(21) Appl. No.: 10/791,209

(22) Filed: Mar. 1, 2004

(51) Int. Cl. C12Q 1/68; G06F 19/00; G01N 33/48; G01N 33/50

(52) U.S. Cl. 435/6; 702/20

(57) ABSTRACT

Methods for detecting a short tandem repeat polymorphism (STRP), such as fragile X syndrome, wherein PCR is used to amplify nucleic acid along the chromosome in the genomic DNA which includes all of the STRs of interest plus a substantial contiguous segment of the nucleic acid adjacent to the STRs. Single-stranded product is then obtained, and colorimetric-labeled oligonucleotides which target for (i) STRs and (ii) the contiguous DNA segment are hybridized with this single-stranded product which is then bound to a solid phase and separated from the remainder of the target material. The labeled oligonucleotide target material is recovered by treatment with base and then hybridized to a microarray having a plurality of spots containing suitable oligonucleotide probes complementary thereto. Following hybridization, colorimetric intensities of the hybridized labeled target material present at specific spots on the microarray are measured to obtain individual values which are compared with results from known control samples to accurately quantify the number of STRs in the region of interest of the DNA being analyzed.

### FIELD OF THE INVENTION

This invention relates generally to diagnostic assays for inherited or sporadic genetic defects, more particularly to assays for diseases or defects caused by short tandem repeats (STRS) and still more particularly to an assay for the genetic defect that causes the fragile X syndrome in persons, fetuses and embryos. These assays of STRPs employ the polymerase chain reaction (PCR) followed by hybridization to a microarray and analysis.

### BACKGROUND OF THE INVENTION

Eukaryotic DNA has tandem repeats of very short simple sequences termed short tandem repeat polymorphisms (STRPs). Repeat polymorphisms include dinucleotide, trinucleotide and tetranucleotide repeats. Trinucleotide and tetranucleotide repeats are repeats of three and four nucleotides. A growing number of diseases are known to be associated with the expansion of trinucleotide STRs (Trottier, Y. et al., Current Biology 3:783-786 (1993); Bates, G. et al., Bioassays 16:277-284 (1994); Kawaguchi, Y. et al., Nature Genetics 8:221-227 (1994)). In these diseases, the size of the repeat block generally correlates with and thereby indicates the severity and age of the onset of the disease. Some diseases are correlated with small increases in the size of the repeat block, for example, Huntington's disease, spino-cerebellar ataxia type I, spinal and bulbar muscular atrophy, Machado-Joseph disease and dentatorubralpallidoluysian atrophy. Other diseases may involve up to a 100-fold expansion of the normal STR, such as fragile X type A, fragile X type E and myotonic dystrophy.

Certain diagnostic and forensic assays have been proposed which are based on the amplification and detection of highly polymorphic classes or repetitive DNA that are present in the human genome, e.g. Craig et al., J. Forensic Sci., Vol. 33, pgs. 1111-1126 (1988); Edwards et al., Genomics, Vol. 12, pgs. 241-253 (1992); Boerwinkle et al., Proc. Natl. Acad. Sci., Vol. 86, pgs. 212-216 (1989); Tautz, Nucleic Acids Research, Vol. 17, pgs. 6463-6471 (1989); and the like. Typically, a segment of DNA that contains the repeated sequence is amplified by polymerase chain reaction (PCR) and then sized by denaturing polyacrylamide gel electrophoresis.

This approach targets the so-called “short tandem repeat” or “STR” repetitive DNA, which is of particular interest for diagnostic and mapping applications because of its size and genomic distribution. The length of the repeated unit in this class of DNA is typically from 2 to 6 nucleotides making them convenient targets for PCR amplification and electrophoretic separation. International Application WO 94/03638 discloses methods for simultaneously amplifying one or more chromosome-specific STRs, for separating the amplified STRs by size electrophoretically and for determining the respective quantities of amplified STR DNA present; the method is used to determine aneuploidy of a selected chromosome. U.S. patent application No. 2003/0224380 suggests that oligonucleotides corresponding to flanking regions of these repeats may be used as primers for the polymerase chain reaction (PCR) on a small sample of DNA, citing Saiki, Science 239:484491 (1988). By amplifying the DNA with labeled, e.g., radioactive or fluorescent, nucleotides, the sample may be resolved on a sequencing gel and visualized by known methods, e.g., autoradiography or fluorescence detection. Because these polymorphisms are comprised of alleles that may differ in length by only a few base pairs, it is indicated that they generally may not be detectable by conventional Southern blotting as used in traditional RFLP analysis. International Application No. WO 94/03638 states that aneulploidy may be detected using amplified STRs of at least 3 nucleotides. The amplified DNA is separated by size electrophoretically, and relative concentrations are determined through the use of fluorescent labels that are spectrally resolvable.

There are a fair number of genetic diseases that fall in the category of STRPs. Included among these are: Myotonic dystrophy-CTG repeat: Huntington Disease and Spinocerebellar ataxia-CAG repeat; and fragile X-CGG repeat. The fragile X syndrome, which is an X-linked dominant disorder with reduced penetrance, is one of the most common forms of inherited mental retardation. This condition afflicts approximately 1 in 1250 males and 1 in 2000 females. The cognitive, behavioral, and physical phenotype varies by sex, with males being more severely affected because of the X-linked inheritance of the mutation.

As the name implies, fragile X is an X chromosome-linked condition. The fragile X phenotype is characterized by a visible constriction near the end of the X chromosome, at locus q27.3, and there is a tendency for the tip of the X chromosome to break off under certain conditions in tissue culture. Researchers have identified the genomic region associated with this condition, and the DNA sequences related to fragile X syndrome (FRAXA gene) are set forth in U.S. Pat. No. 6,197,500. The disorder-causing mutation results in amplification of a CGG repeat in the 5′ untranslated region of FRAXA located at Xq27.3. The fragile X-CGG repeat has four forms: common (6-40 repeats), intermediate (41-60 repeats), premutation (61-200 repeats), and full mutation (>230 repeats). The mutation that ultimately results in the fragile X phenotype generally occurs in stages. In the early stages, the gene is not fully defective, rather there is the “pre-mutation”, and the carrier of the permutation may have a normal phenotype. However, a mutation can occur in carrier females that may produce the phenotype in their offspring. The consequences of the increased number of CGG repeats range from abnormal behaviors to mental retardation. The number of CGG repeats above the normal range (6 to 40) determines the severity of the syndrome.

For many years, the only way to diagnose the fragile X syndrome was via microscopic examination of an afflicted individual's chromosomes after cell growth and treatment in tissue culture. In such an examination, a laboratory would examine the X chromosome to ascertain it had broken a tip. Some early attempts to develop PCR-based methods to directly identify the CGG repeat sequence at the genomic level were unsuccessful (see E. J. Kremer, “Mapping of DNA Instability at the Fragile X to a Trinucelotide Repeat Sequence p(CGG)n”, Science, vol. 252, Jun. 21, 1991, pp. 1711-1714.)

More recent methods of diagnosis have used PCR and gel electrophoresis separation to achieve identification based on molecular weights. For example, U.S. Pat. No. 5,213,961 to Bunn et al. discloses a method of quantitative PCR by competitive methodology, wherein the parameters affecting DNA amplification and a mechanism to distinguish differences in template (both test and control) ratios and copy numbers are discussed. It is mentioned that it might be used to detect somatic cell mutations. Bunn et al. address the effect of various parameters on the amplification process which arise predominantly from the nature of the DNA primers and their respective primer binding sites; however, the system is limited to use of a standard that is sufficiently close to the target that the target and sample are co-amplified at the same rate by PCR. Moreover, the standard must differ from the target such that its length can be later altered by enzymatic action, thus allowing the standard and target to be separated and quantified by electrophoresis.

U.S. Pat. No. 6,180,337 to Caskey describes a method of measuring and comparing the expression of the FMR-1 gene in normal and unaffected individuals wherein variation in the expression in affected individuals compared with that in normal individuals indicates a mutation for the fragile-X syndrome. The method attempts to quantify unstable mRNAs, instead of stable genomic DNAs, which may often lead into an inaccurate diagnosis. U.S. Pat. No. 6,143,504 to Das uses methylation-specific PCR in order to identify males having fragile X syndrome. However, this method is truly limited to diagnosing full mutations having more than 200 repeats. U.S. Pat. No. 6,197,500 to Sutherland, which describes a purified and isolated DNA molecule comprising the human fragile X locus, teaches detecting PCR-amplified products of a CGG repeat by hybridizing with a probe complementary to the amplified product which was used to characterize the genomic DNA. However, it does not teach how to specifically quantitate the number of repeats in a manner necessary to achieve a subsequent adequate diagnosis; it merely suggests employing a probe that will hybridize under appropriate stringency to the abnormal sequence. U.S. Pat. Nos. 5,658,764 and 6,200,747 disclose a method for detecting fragile X syndrome which uses PCR, employing an analog of dGTP, followed ultimately by gel electrophoresis.

However, these methods possess only a limited ability to determine the number of CGG repeats and thus to provide accurate diagnosis. This is generally due to difficulty in PCR of amplifying regions of CGG repeats; often not enough PCR products are produced to permit accurate gel electrophoresis analysis which requires a significant quantity for detection.

Thus, the search has continued for an accurate assay for diseases and defects resulting from STRPs, and particularly for fragile X syndrome, that is straightforward and reliable.

### SUMMARY OF THE INVENTION

A method using highly sensitive colorimetric detection has now been developed that is able to accurately estimate the copy number of STRs present in genomic DNA, e.g. CGG repeats in the 5′-untranslated region of the FRAXA gene. A DNA region is selected that contains the STRs and a contiguous region or segment that serves as an internal control, so that they are coamplified from a sample of genomic DNA using PCR. For fragile X syndrome, the DNA region encoding the internal control is selected so that it is located on the X-chromosome either 5′ or 3′ of the CGG repeats region; so long as the CGG repeats region and this internal standard segment are contiguous, they will always be co-amplified. Following PCR amplification of the sample, appropriate steps are taken to obtain the single-stranded product. Both labeled CCG target and labeled internal standard target are then provided, and these labeled targets are hybridized to the single-stranded, PCR-amplified product. After washing to remove non-hybridized target, the remaining labeled oligonucleotide targets which hybridized to the PCR products are obtained, and they are quantified by subsequent hybridization to a microarray containing a CGG probe and an internal control probe. The copy number of the CGG repeats of such an unknown sample is then accurately estimated by determining the ratio of the signal intensity at the CGG repeat region probe to that at the internal control probe and comparing such ratio with values that were earlier generated from known control samples. Similar analyses for other STRPs are carried out by appropriately selecting an adjacent segment of the relevant chromosome for use as an internal control and coamplifying it and the STR region.

In one particular aspect, the invention provides a method for detecting a mutation indicative of fragile X syndrome, which method comprises the steps of (a) obtaining genomic DNA to be tested, (b) using PCR to amplify nucleic acid along the X-chromosome in the genomic DNA which includes all of the CGG repeats of the untranslated portion of the FRAXA gene plus a substantial contiguous segment of nucleic acid adjacent to said CGG repeats, (c) obtaining single-stranded product from the amplified nucleic acid of step (b), (d) hybridizing colorimetric-labeled oligonucleotides which target for (i) (CGG) repeats and (ii) said contiguous nucleic acid segment with said single-stranded product of step (c), (e) binding said single-stranded product of step (c) to a solid phase, (f) separating said hybridized product of step (d) from the remainder of the target material, (g) recovering the labeled target material from the separated product of step (f), (h) then hybridizing the recovered labeled target material of step (g) to a microarray having a plurality of spots containing suitable oligonucelotide probes complementary thereto, (i) following hybridization to the microarray, measuring the colorimetric intensities of the hybridized labeled target material present at specific spots on the microarray to obtain individual values therefor, and (j) comparing the results of step (i) with results from known control samples to accurately quantify the number of CGG repeats in the FRAXA gene of the obtained genomic DNA.

In another particular aspect, the invention provides a method for detecting a mutation indicative of fragile X syndrome, which method comprises the steps of (a) obtaining genomic DNA to be tested, (b) using PCR and forward and reverse primers to amplify nucleic acid along the X-chromosome in the genomic DNA which includes all of the CGG repeats and a contiguous portion of the translated FRAXA gene, said forward primers having an anchoring moiety at the 5′ end thereof, (c) purifying the double-stranded product of step(b), (d) obtaining single-stranded product from step (c) by digesting the antisense strand thereof with an exonuclease, (e) hybridizing the product of step (d) with fluorescence-labeled antisense targets for (CGG) repeats and for the contiguous portion of the FRAXA gene, (f) separating said hybridized product of step (d) from the remainder of nonhybridized targets by binding to a solid phase through said anchoring moieties at the 5′ ends of said forward primers, (g) hybridizing the product of step (g) to a microarray containing suitable probes and, following hybridization to said microarray, measuring the fluorescent intensities of fluorescence-labeled target material present to obtain individual values therefore, and (h) comparing the results of step (g) with results from known control samples using the following formula: N=30 +(A−1.03)66.4 where N is the number of repeats and A is the ratio of the FI of the target which hybridized with CGG probes to the FI of the target which hybridized to the probes for the contiguous segment, to accurately quantify the number of CGG repeats in the FRAXA gene of the DNA obtained.

In a further particular aspect, the invention provides a method for detecting a short tandem repeat polymorphism (STRP), which method comprises the steps of (a) obtaining genomic DNA to be tested, (b) using PCR to amplify nucleic acid along the chromosome in the genomic DNA which includes all of the STRs of interest plus a substantial contiguous segment of the nucleic acid adjacent to said STRs, (c) obtaining single-stranded product from the amplified DNA of step (b), (d) hybridizing colorimetric-labeled oligonucleotides which target for (i) STRs and (ii) said contiguous nucleic acid segment with said single-stranded product of step (c), (e) binding said single-stranded product of step (c) to a solid phase, (f) separating said hybridized product of step (d) from the remainder of the labeled target material, (g) recovering the labeled target material from the product of step (f), (h) then hybridizing the recovered labeled target material of step (g) to a microarray having a plurality of spots containing suitable oligonucleotide probes complementary thereto, (i) following hybridization to the microarray, measuring the colorimetric intensities of the hybridized labeled target material present at specific spots on the microarray to obtain individual values therefor, and (j) comparing the results of step (i) with results from known control samples to accurately quantify the number of STRs in the region of interest of the obtained DNA.

In a yet further particular aspect, the invention provides a kit to detect a mutation indicative of fragile X syndrome, which kit comprises: (a) a pair of DNA oligonucleotides that will function as forward primers and reverse primers in a polymerase chain reaction (PCR) for amplifying mammalian genomic DNA, wherein the forward primer is complementary to a 3′ nucleotide sequence of the antisense strand of the X-chromosome at a location therealong which is 5′ of the untranslated region of the FRAXA gene and the reverse primer is complementary to a location within the FRAXA gene or 3′ thereof, said forward primer having an anchoring moiety covalently linked to the 5′ end thereof, said reverse primer having phosphate at its 5′ end, and said pair of primer oligonucleotides being specific to amplify the region of genomic DNA which contains all of the CGG repeats and a substantial contiguous segment that serves as an internal control, (b) labeled oligonucleotides which separately target said CGG repeat region and said internal control segment, (c) buffers and enzymes for carrying out (i) a PCR, (ii) digestion of the antisense strand, (iii) DNA-DNA hybridizations and washing, (iv) dissociation of hybridized labeled oligonucleotide targets; and (v) colorimetric quantitation, (d) at least one microarray having a plurality of spots, which spots each have attached thereto DNA probe complementary to one of said labeled oligonucleotide targets; and (e) means for performing diagnosis for the number of CGG repeats using the results of colorimetric scanning of said microarray and earlier generated data from control samples.

In a still further particular aspect, the invention provides a kit to detect a mutation indicative of STRP which kit comprises (a) a pair of DNA oligonucleotides that will function as forward primers and reverse primers in a polymerase chain reaction (PCR) for amplifying mammalian genomic DNA, said pair of primer oligonucleotides being specific to amplify a selected region of genomic DNA which contains all of the STRs and a substantial contiguous segment that serves as an internal control; wherein the forward primer is complementary to a 3′ nucleotide sequence of the antisense strand of the selected region of the chromosome and the reverse primer is complementary to the 3′ end of the sense strand of the selected region, said forward primer having an anchoring moiety covalently linked to the 5′ end thereof, and said reverse primer having its 5′ end blocked to elongation, (b) labeled oligonucleotides which separately target said STR region and said internal control segment, (c) buffers and enzymes for carrying out (i) a PCR, (ii) digestion of the antisense strand, (iii) DNA-DNA hybridizations and washing, (iv) dissociation of hybridized labeled oligonucleotide targets; and (v) colorimetric quantitation, (d) at least one microarray having a plurality of spots, which spots each have attached thereto DNA probe complementary to one of said labeled oligonucleotide targets, and (e) means for performing diagnosis for the number of STRs using the results of colorimetric scanning of said microarray and earlier generated data from control samples.

### DETAILED DESCRIPTION OF THE PREFERRED EMBODIMENTS

The following definitions of terms and acronyms are provided to better understand the detailed description set forth hereinafter.

Hybridization is used to denote the formation of a duplex structure between complementary strands of DNA carried out either in solution or in a solid phase, wherein one of the two strands is immobilized onto a solid surface or matrix.

Annealing is used herein to mean incubation of a single-stranded or heat-denatured duplex nucleic acid analyte with an oligonucleotide probe or primer, under hybridization conditions enabling the probe or primer to bind to its complementary sequence within the analyte nucleic acid, either at a slowly decreasing temperature or at a single temperature.

Analyte or analyte nucleic acid is used to describe a compound in a DNA sample which is the object of analysis.

Label or detectable label (or “tag”) refers to a substituent that can be attached to a nucleic acid sequence which enables its detection and/or quantitation. Examples include radiolabels such as ³²p, ³³p, and ³⁵S; colorimetric indicators, such as fluorescent, chemiluminescent or colored compounds; ligands such as biotin; and chemical groups that are distinguishable by mass or other spectroscopic properties. More specific examples of suitable labels include xanthine dyes, rhodamine dyes, naphthylamines, benzoxadiazoles, stilbenes, pyrenes, acridines, Cyanine 3 and Cyanine 5. A label may be introduced into analyte nucleic acid by a variety of means, including chemical reaction, incorporation of labeled nucleotide by enzymatic reaction (including polymerase, kinase or ligase), or by hybridization or annealing of a labeled probe with the analyte nucleic acid.

Probe refers to a nucleic acid sequence used as a reagent to bind its complementary sequence determined by the analyte nucleic acid via a hybridization reaction.

Sequence means a string of bases within a nucleic acid comprising A, G, C, T residues in DNA, linked together in a specific order and chain length.

Complementary or complementary sequence refers to two sequences that are capable of forming a two-stranded (duplex) structure.

Labeled probes is herein used to mean an oligonucleotide bearing one or more detectable labels or tags, which is capable of binding to its complementary sequence within a nucleic acid analyte, enabling its detection and/or quantitation.

Capture probe is used herein to mean an oligonucleotide of specific sequence bound at one end (i.e. tethered) to a solid surface, enabling the capture of a nucleic acid or oligonucleotide containing a complementary sequence onto the solid surface in a hybridization reaction.

Target, target sequence, target strand or target nucleic acid is used herein to refer to a nucleic acid sequence whose presence is the object of detection, for example, through hybridization with a specific DNA probe. The term “target sequence” is sometimes used in a broad sense to mean the nucleic acid molecule or fragment bound by a DNA probe, or in a restricted sense to mean a specific nucleotide sequence derived from the target nucleic acid which binds to the DNA probe via complementary base pairing.

Tethered, or surface-tethered is used herein to refer an oligonucleotide DNA probe that is bound at one end with some surface, through a covalent bond or otherwise strong bond formed between a functional group on the surface and a functional group at one end of the DNA probe.

Solid phase hybridization means a hybridization reaction conducted in which one of the two “reactant strands” participating in formation of a duplex structure is immobilized on a solid support.

Bordering or flanking sequence is used to refer to nucleic acid segments that are near, adjacent to and/or include the ends of a selected DNA sequence and a chromosome.

Oligonucleotide means a short DNA strand, which can be chemically synthesized, typically of a length up to about 100 nucleotides.

Gene means a unit of genetic function, including sequences encoding a protein, intronic (noncoding) sequences interpersed within a gene, and additional sequences functioning in the regulation of the gene.

Genome means the entire complement of genes, intergenic sequences and other genetic elements that comprise an organism or autonomously replicating entity. Microarray or DNA chip means a two-dimensional array of surface-tethered DNA probes formed on a surface, enabling simultaneous analysis of a multiplicity of hybridization reactions, typically in a miniaturized format, with individual DNA probes arrayed at center-to-center spacing of less than one millimeter.

Primer means an oligonucleotide possessing a free 5′-OH terminus, which will base-pair with a “template strand” and thus can be elongated by a polymerase enzyme. For example, an oligonucleotide primer annealed with a DNA template can serve as a substrate (along with deoxynucleoside 5′-triphosphates) for a DNA polymerase, resulting in “primer extension,” as in the PCR reaction. Primer pair means two primers that bind to opposite strands of a nucleic acid segment.

PCR fragment means a fragment of DNA of defined length (defined by the spacing between priming sites on the template) formed by the polymerase chain reaction.

Denature or denatured means separation (dissociation) of the two strands of a duplex nucleic acid molecule under conditions which destabilize the double helix, most commonly, elevation of temperature (“heat-denaturation”). Repeat or repetitive sequence means a sequence of short repeat sequences, particularly CGGCGGCGG.

5′-end/terminus means the end of a nucleic acid chain containing a nucleotide with a non-esterified carbon-5 on its deoxyribose.

3′-end/terminus means the end of a nucleic acid chain containing a nucleotide with a non-esterified carbon-3 on its deoxyribose.

CCD—charge coupled device.

CI—colorimetric intensity.

FI—fluorescence intensity.

PCR—polymerase chain reaction.

SSC-standard saline citrate, a solution containing 150 mM sodium chloride and 15 mM sodium citrate.

STR—short tandem repeat.

STRP—short tandem repeat polymorphism.

Applicant's diagnostic method utilizes PCR to initially amplify specific DNA sequences that are present in low abundance relative to total genomic DNA. By using PCR, a specific DNA sequence can be amplified one hundred thousand fold or more to facilitate its detection it is when present in the starting DNA.

U.S. Pat. Nos. 4,683,202, 4,683,195, 4,800,159 and 4,965,188, the disclosures of which are incorporated herein by reference, provide details of the now well known PCR process which is utilized by the present invention. PCR amplifies a DNA sequence several orders of magnitude in a few hours through the use of oligonucleotide primers complementary to sequences flanking a particular region of interest to effect primer-directed DNA synthesis in opposite and overlapping directions. By employing repeated cycles of high temperature template denaturation, oligonucleotide primer reannealing, and polymerase-mediated extension, DNA sequences can be faithfully amplified several hundred-thousand fold. Generally PCR requires knowledge of the sequence of both the 5′ and the 3′ end of the template being amplified so that two different primers for each template may be designed, one forward primer for generating the sense strand and one reverse primer for generating the antisense strand.

The PCR process in the present invention preferably uses a single pair of primers that include oligonucleotides that are capable of acting as points of initiation of desired DNA synthesis. The oligodeoxynucleotide primers each possess a free 3′ OH group which, upon hybridization to a nucleic acid template, is recessed relative to the 3′ end of the desired template and thus acts as a site of initiation of the synthesis of polymerization of a nucleic acid polymer, the sequence of which is complementary to the template strand, in the presence of (a) deoxyribonucleotide substrates, (b) an appropriate enzyme capable of effecting DNA replication, and (c) a suitable temperature and buffers to provide desired pH. Primers are preferably produced synthetically for expediency. PCR typically employs two primers that bind to a selected nucleic acid template, each of which is complementary to one of the two 3′ ends or borders of the duplex segment to be amplified. They are commonly referred to as forward and reverse primers. The primers are combined with the other PCR reagents under conditions that induce primer extension, i.e., with four different nucleoside triphosphates (or analogs thereof), an appropriate polymerase and an appropriate buffer (“buffer” includes agents for determining pH and ionic strength, cofactors, etc.) at a suitable temperature. In a PCR method where the polymerase is Taq polymerase, the buffer may contains 1.5-2 mM of a magnesium salt, preferably MgCl₂, 15-200 μM of each nucleoside triphosphate, 1 μM of each primer and e.g. 50 mM KCl, 10 mM Tris buffer at pH 8.4, and 100 μg/ml gelatin. Such kits for performing PCR amplification are commercially available from numerous vendors.

Each primer should be sufficiently long to initiate or prime the synthesis of extension DNA products in the presence of an appropriate polymerase and other reagents, such as those mentioned above. Appropriate primer length is dependent on many factors, as is well known; typically, in the practice of applicant's method, a primer will be used that contains 15-30 nucleotide residues. Short primer molecules generally require lower reaction temperatures to form and to maintain the primer-template complexes that support the chain extension reaction.

The primers used need to be substantially complementary to the nucleic acid containing the selected sequences to be amplified, i.e, the primers must bind to, i.e. hybridize with, nucleic acid containing the selected sequence (or its complement). The primer sequence need not be entirely an exact complement of the template; for example, a non-complementary nucleotide fragment or other moiety may be attached to the 5′ end of a primer, with the remainder of the primer sequence being complementary to the selected nucleic acid sequence. Primers that are fully complementary to the selected nucleic acid sequence are preferred and typically used.

Generally, any specific nucleic acid sequence can be amplified by the PCR process so long as a sufficient number of bases at both ends of the sequence are known in sufficient detail so that two oligonucleotide primers can be prepared which will hybridize to different strands of the desired sequence that are located at the desired relative positions along the nucleic acid sequence. As a result, the extension product synthesized from one primer, following its separation from its template (complement), will serve as a template for extension of the other primer into a nucleic acid of defined length in the next cycle.

In a preferred embodiment of the present invention to diagnose for a STRP, a pair of primers are used. One of the primers, i.e., the forward primer, is complementary to a sequence which is near, abuts and/or includes the 3′ end of the antisense of the selected DNA region. The other primer, i.e., the reverse primer, contains the complement of the sequence which is near, abuts or and/or includes the complement of the sequence at the 3′ end of the selected DNA region.

Depending upon the STRP to which the assay will be directed, a decision is first made as to the length of the nucleic acid on the relevant chromosome to be amplified. As a result of the completed sequencing of the human genome, the sites of these STRPs are now known, and thus attention is directed to the locus where the site appears. Judgment is then employed in selecting flanking sequences that will include all of the STR segment and a contiguous segment either 3′ or 5′ of the STR segment that will serve as an internal control. For fragile X syndrome, this election of a contiguous sequence that is 3′ of the STR region is preferred, although an alternative region 5′ thereof might be employed. Once the length of the nucleic acid segment to be amplified is selected, suitable oligonucleotides primer pairs are designed that will effect the co-amplification of the entire STR region and the selected contiguous segment to be used for an internal control. The primers will be designed to be complementary to the 3′ regions of each of the strands of the nucleic acid which regions are near, abut and/or include the particular end of the selected nucleic acid segment.

Generally, primers will be between about 15 and 30 nucleotides in length and preferably between about 18 and 27 nucleotides in length. They are preferably chosen to hybridize to a unique DNA sequence in the genome so as to maximize the desired location hybridization that will occur.

The length of nucleic acid along the chromosome in question that will be amplified will of course be determined by the length of the STR region as it is this variation in length toward which the assay is directed. Generally, a nucleic acid length is selected which would be between about 350 nucleotides and 2000 nucleotides in a normal human chromosome, and more preferably a nucleic acid length of a between about 500 nucleotides and 1000 nucleotides is selected.

To facilitate the assay, a kit is provided which includes all of the necessary tools. For example, a kit to detect a mutation indicative of fragile X syndrome should include a pair of DNA oligonucleotides that will function as forward primers and reverse primers in a polymerase chain reaction (PCR) for amplifying mammalian genomic DNA, wherein forward primer is complementary to a 3′ nucleotide sequence of the antisense strand of the X-chromosome at a location therealong which is 5′ of that untranslated region of the FRAXA gene where the CGG repeats are located and wherein the reverse primer is complementary to a location within the FRAXA gene, i.e, 3′ of the repeat region. The forward primer should have an anchoring moiety covalently linked to the 5′ end thereof, when the reverse primer has phosphate at its 5′ end. This arrangement might be reversed if desired. Such pair of oligonucleotides are thus specific to amplify the region of genomic DNA which contains all of the CGG repeats and a substantial contiguous segment that serves as an internal control. Labeled oligonucleotides which separately target the CGG repeat region and the internal control region are provided, along with buffers and enzymes for carrying out (i) a PCR, (ii) digestion of the antisense strand, (iii) DNA-DNA hybridizations and washing, (iv) dissociation of hybridized, labeled oligonucleotide targets; and (v) calorimetric quantitation. The kit would include a microarray having a plurality of spots, which spots each have attached thereto DNA probe complementary to one of the labeled oligonucleotide targets. Further included is means for performing diagnosis for the number of CGG repeats using the results of scanning, e.g., calorimetric, of the microarray, which diagnosis is based upon earlier generated data from control samples.

The forward primers of the pair of primers that are used preferably have an anchoring moiety covalently linked to the 5′ end of each primer. The reverse primers are derivatized with phosphate at the 5′ ends. Advantage is taken of this anchoring moiety in a separation step in the assay as explained hereinafter. Generally, any anchoring moiety can be used that will serve to couple the oligonucleotide to a solid surface or solid phase.

As well known in this art, various solid phase material can be used; for example, the solid support material can be selected from any of a wide variety of materials that are commonly used, such as those which are commercially available from Amersham Biosciences, BioRad, and Sigma. It can be in the form of particles, plates, matrices, fibers or the like, and it may be made of silica, cellulose, agarose beads, controlled-pore glass, polymeric beads, gel beads, or magnetic beads. Magnetic beads are preferred because the use of such facilitates their subsequent separation from the supernatant by the straightforward application of a magnetic field. Such can be done using flow chambers or by simply pipetting. Such magnetic beads, for example those sold as Dynal beads or those sold by Advanced Magnetics, can be used to separate the amplified DNA from the remainder of the biological sample and the PCR material and reaction products by washing. This same property is also used to advantage in separating decoupled target material, at a later stage in the assay procedure. Although the particles in bead form are preferred for facility and handling, other shaped particles or substrates might alternatively be employed. Such commercially available magnetic beads are generally small nonporous spheres that are coated with a layer of magnetite to provide the desired magnetic properties, and then with an exterior coating. Magnetic beads, which are commercially available for these purposes are produced in various ways; often paramagnetic metals, such as metal oxides, are encapsulated with a suitable coating material, such as a polymer or a silicate, to produce coated beads that are about 1 μm-100 μm in diameter.

Anchoring moieties and coupling agents which are complementary and bind to each other are used as a linkage to attach the amplified DNA to such solid support. Many varieties of binding pairs are well known in the art and may be suitably employed. The anchoring moiety may join directly to the solid phase or, more usually, to a complementary coupling agent carried by the solid phase. A preferred binding system employs avidin or streptavidin and biotin. Streptavidin, for example, is covalently attached to the exterior surface of the solid support, e.g., the magnetic beads, and it, in turn, binds strongly to biotinylated DNA. Such magnetic beads suitable for applications of interest are commercially available from a number of vendors. Beads which have streptavidin bound to the surface of the beads, having a nominal size of about 1 micron in diameter, are sold by Active Motif of Carlsbad, Calif. Other binding pairs, e.g. antibody-antigen and the like, may alternatively be used as such an intermediate linkage. Such derivatized beads may be supplied as an optional part of the kit along with buffer to facilitate the washing as set forth in item (iii) above.

The other items that are supplied as a part of the categorized portion of the kit are well known items which are commercially available and commonly included as part of any PCR kit. They are described in detail in the group of four U.S. references which provide details of the now well known PCR process.

A key item of the kit is the set of labeled oligonucletoides which are designed to separately target the CGG repeat region and the region that was selected as the internal control. The labels used may be any of those items that have commonly been used, selected from the wide range of materials commercially available for labeling nucleic acids, including indicator dyes, radionuclides, antibodies, enzymes and the like. Preferably, the label is a calorimetric indicator, and more preferably a fluorescent dye for simplification of the final assay; however alkaline phosphates, peroxides, β-galactosidase (beta-galactosidase) and haptens, such as digoxin and digoxygenin, as well as items as chemiluminescent moieties may be used. Although a short linker may be used so that the label will not interfere with hybridization of the target as is well known in this art, generally the label is connected directly to the target. The preferred calorimetric indicator is a Cy-3 fluorescent dye.

Of the two separate, labeled, oligonucleotide targets, one target is designed to hybridize to the STR section. These oligonucleotides should include about 3 and 7 triplets and preferably between about 4 and 6 triplets, i.e., being between about 12 and 18 nucleotides in length. The labeled target material for the internal control is generally between 21 and 54 nucleotides in length and preferably between about 30 and 45 nucleotides in length. It is simply chosen so as to be complementary to the sense strand of the nucleic acid contiguous segment chosen, e.g. a segment of the translated FRAXA gene. The amount of labeled STR target is preferably at least about 5 times the amount of internal control target, more preferably at least about 10 times as much, and most preferably at least about 20 times as much.

The kit also contains a microarray having a plurality of microspots which have selected DNA probes attached thereto; the probes are complementary to one of the labeled targets. Although any of the myriad of developed arrays for labeled DNA targets can be used, including those two-dimensional assays wherein probes for targets are bound directly to a flat substrate or in a well of microtiter plate; it is preferred to provide a three-dimensional biochip, such as those described in U.S. Pat. No. 6,174,683 and in published international application WO 02/059372, entitled “Three Dimensional Format Biochips.” In such a three-dimensional array, the probes are not connected to the solid surface of a well in a plate or to a glass slide or other plate, but they are instead presented in three-dimensional array by attachment to microspots of polymerized hydrogel. This arrangement isolates the probes from the solid substrate and presents an expanded surface area for presentation of the probes and for the ultimate capture of the labeled target molecules. Preferably, a plurality of such 3-D microspots are provided on each glass slide or in each well of a microwell plate, for each of the different targets employed in the assay.

In the preferred embodiment where the anchoring moieties are covalently attached to the forward primers that will then be incorporated in the sense strands, the probes are simply sections of the nucleic acid sense strand originally selected; they are derivatized so as to couple or bind to the microarray plate or in the preferred arrangement to the hydrogel spots that are adhered to the microarray plate. Any suitable linker may be used and preferably a C-6 amino linker is employed as is generally known in this art. The probe for the STR section should contain a plurality of CGG triplets, preferably between about 6 and 20 triplets and more preferably between about 8 and 15 triplets; there are preferably at least about twice as many triplets in the probe as there are in the target material. The oligonucleotide probe for the internal control target material is likewise simply a segment of the sense strand of the nucleic acid from the contiguous region chosen of suitable length. It is preferably at least as long as the target material and is preferably of about the same length as that herein indicated for the internal control target material.

As above mentioned, the kit would include appropriate chemicals to facilitate the hybridization reaction. Following incubation of the hybridization solution with the slides or wells, washing is carried out to remove unbound labeled target material. The resulting slide can be observed in any suitable manner, as by using a fluorescence detector when fluorescent dyes are used. Other appropriate detectors would of course be alternatively used depending on the nature of the particular label chosen for attachment to the targets. An appropriate algorithm or other means that is provided in the kit is then used to interpret the results of the colorimetric scanning of the microarray; such is developed and based upon earlier generated data from control samples.

As an example of an assay procedure to detect the presence of a STRP, a small amount of an unknown genomic DNA is first obtained; for example, about 10 ng is considered sufficient. This unknown sample is subjected to PCR in a reaction chamber having a volume of 50 microliters. About 1 μM each of forward and reverse primers for selected nucleic acid segment including the STR section and the chosen contiguous segment of nucleic acid are added along with the ingredients of a commercially available PCR system, that would contain appropriate of deoxyribonucleoside substrates, an appropriate enzyme and buffers. Generally, between about 5 and about 40 temperature cycles of the PCR replication process are carried out to create a desired amount of the amplified nucleic acid sequence chosen. Upon completion of PCR amplification, the double-stranded product is purified, using standard means to remove the unreacted primers and nucleotide substrates and the like. Such purification kits are commercially available, and elution is then carried out with an appropriate amount of DI water.

The antisense strand of the purified DNA is then digested with a suitable nuclease such as lambda exonuclease(NEB). Following such digestion, the biotinylated single strand DNA having the nucleotide sequence of the sense strand of the nucleic acid originally chosen remains. It is then mixed with the labeled target material for both the STR section and the internal control segment; about 200 nM of the labeled target oligonucleotide for the STR section and about 10 nM for the internal control section are employed, along with a suitable commercially available buffer system. The mixture is subjected to a denaturation temperature of about 95° C. for about 10 minutes and is then incubated for an appropriate length of time, e.g., a temperature of about 35° C. to 40° C. for about 2 to 5 hours, using continuous shaking or the like to promote hybridization.

Following incubation, the hybridized sample material is immobilized through the biotin anchoring moieties of the amplified DNA strand to streptavidin beads or another suitable solid substrate. Immobilization is effected by incubation in a suitable buffer at room temperature for about 15 minutes with shaking or the like. After providing time for the attachment between the beads and the biotinylated DNA to be completed, the beads are washed to eliminate all of the other components including the labeled targets that have not specifically hybridized. Generally, multiple washings, optionally of increasing stringency, will be employed.

Following these washings, labeled target is eluted from strand bound to the solid support by treatment with 0.1 M NaOH or a comparable base and incubating for about 5 minutes at room temperature. Following elution, the liquid supernatant is separated from the beads, which are being immobilized by magnetic attraction, and it is collected and neutralized. The liquid solution containing the labeled targets is then directly subjected to a suitable microarray.

Hybridization to the microarray is carried out in a solution containing suitable buffers for a period of usually at least about 12 hours at an appropriate temperature e.g. 40° C. to 50° C. Following hybridization, the microarray is washed multiple times using a suitable buffer-containing solution, and it is then subjected to colorimetric analysis. When the labels are fluorescent as is preferred, the intensities of the fluorescent signals that are given off by probes which are specific to the different target material that hybridized with the amplified nucleic acid are recorded; these values provide a quantitative indication of the relative length of that particular STR segment by comparison to the amount of internal standard that was found to have been coamplified in the amplified PCR product, as explained in detail hereinafter.

Certain of the steps in the initial portion of the assay can be performed in various sequences as desired. For example, the biotinylated, amplified double-strand DNA might be first coupled with the streptavidin-carrying magnetic beads before the antisense strand is digested, or alternatively so coupled before the labeled targets are added to the mixture. However, the preferred method of carrying out the assay is as indicated above, wherein the double-stranded product is first treated to digest the antisense strands before the labeled targets, i.e. DNA oligonucleotides which carry fluorescent labels, are added, and mixture is then maintained under conditions conducive to hybridization so that the single strand DNA targets will have hybridized to the complementary sequences of the amplified DNA before attachment is made to the streptavidin-carrying magnetic beads or the like. Once the beads have been washed, they may be treated with alkali, e.g. a sodium hydroxide solution at room temperature to free the fluorescent-labeled synthetic targets. The resulting aqueous solution can then be directly used in a suitable assay for the two targets that hybridized with the amplified DNA sample, but have now been liberated as a result of such alkali treatment; however, it is preferably first neutralized.

Specifics of the invention are now described from the standpoint of an example of analysis designed to focus upon detection of the STRP referred to as fragile X syndrome. To provide tools for interpreting results from unknown samples to be analyzed for fragile X syndrome, the method described above is first used to test two control samples which are known to, respectively, have 30 CGG repeats and 117 CGG repeats. The labels used are fluorescent; thus, the colorimetric intensity that is measured at each respective probe on the microarray is the fluorescence intensity (FI). The following results were obtained:
1. For the control sample that is known to have 30 repeats:
   FI of CGG probe=17,598
   FI of internal control probe=17,008
   (FI of CGG) /(FI of internal control)=1.03
2. For the control sample that is known to have 117 repeats:
   FI of CGG probe=11,005
   FI of internal control probe=4,708
   (FI of CGG) /(FI of internal control)=2.34
      The results obtained from these control samples are proportional, and they provide tools that can be used to determine the number of repeats in any unknown sample using an equation derived from these results. More specifically, it has been found that the results from two such control samples can be used to create an algorithm that will then allow the use of the FI or other comparable calorimetric values later obtained from an unknown sample to compute the number of repeats in such unknown sample.

The following algorithm was developed, based upon test data for STRs in a particular gene, e.g. fragile X (FRAXA) gene, from which a simplified equation is then derived for this particular STRP:

$$N = K + \frac{( A - B ) \times Q}{( C - B )}$$

N=Computed number of STRs in an unknown sample.

K=Number of STRs in control sample with smaller number of repeats.

CI=colorimetric intensity.

A=Ratio of the CI of the STR probe divided by the CI of the internal control probe for unknown sample.

B=Ratio of CI's for the control sample with K repeats.

Q=Difference in number of repeats between the control sample with larger number of repeats and the control sample with the smaller number of repeats.

C=Ratio of the CI of the STR probe divided by the CI of the internal control probe for the control sample with the larger number of repeats.

This algorithm is then used to derive a specific equation for analysis of suspected fragile X syndrome. As a result of the testing of the two control samples having 30 and 117 repeats, values are now known for K, B, C and Q; thus, the algorithm can be simplified to the following equation for fragile X:
N=30+(A−1.03)66.4.

To test the validity of this analysis, an unknown sample is then run. The details of this assay of such unknown are set forth hereinafter in the Example that follows; however, they should be considered to be illustrative and not limiting of the invention, the scope of which is defined in the claims appended hereto.

### EXAMPLE

PCR was performed in a total volume of 50 μl containing 10 ng of genomic DNA and 1 μM each of primers for a selected nucleic acid segment of the X-chromosome in the locus of the FRAXA gene sequence. The selected segment includes the entire CGG repeats section and a 3′ internal control segment which is contiguous thereto. The GC-Rich PCR System from Roche is used. The FRAXA forward and reverse primers that are used are oligonucleotides having nucleotide base sequences SEQ ID NOS: 1 and 2 (see TABLE). The forward primer is 21 nucleotides in length, and the reverse primer is 27 nucleotides in length. They span a total gene segment which is at least 254 nucleotides in length in the “normal” X-chromosome. The forward PCR primers were 5′ biotinylated, and the reverse primers were 5′ phosphorylated. The PCR temperature cycle conditions used were: 95° C. for 2 min, followed by 25 cycles at 95° C .for 1.5 min, 56° C. for 1 min, and 72° C. for 2 min. Final extension was performed at 72° C. for 7 min.

The amplified DNA was purified with the QIAquick PCR purification kit (Qiagen) and then eluted with 50 μl of DI water. The antisense strand of the purified DNA was then digested with lambda exonuclease (NEB).

The remaining biotinylated single-strand DNA was hybridized to two different 5′-Cy3-labeled target oligonucleotides: Biocept #3584 (SEQ ID NO: 3) including (CCG)n, and Biocept #3595 (SEQ ID NO: 4) complementary to the chosen 3′ FRAXA internal control. Hybridization was carried out in 1×B&W buffer (5 mM Tris-HCl, pH 8, 0.5 mM EDTA, 1 M NaCl). The sample was denatured at 95° C. for 10 min, followed by incubation at 37° C. for 3 hr in a shaker.

The hybridized target material was then immobilized by coupling the biotinylated sense strands to streptavidin beads (Active Motif) by incubating in 1×B&W buffer at room temperature for 15 min on a shaker. The DNA/bead complex was washed 3 times with 1×B&W, and the labeled target oligonucleotides were then separated from the bound single-strand DNA by adding 0.1 M NaOH and incubating at room temperature for 5 min. The supernatant containing the released, labeled target oligonucleotides was collected and neutralized.

The labeled target oligonucleotides were then hybridized to a HydroArray microarray containing two probes: Biocept #3594 (SEQ ID NO: 5) and Biocept #3686 (SEQ ID NO: 6). These probes were complementary, respectively, to the labeled targets for the FRAXA internal control and CGG repeats (see TABLE). Hybridization at the microarray was carried out in a solution containing 3×SSC and 0.1% Triton X-100 for about 14 hours at 45° C. The array was washed 3 times in a solution containing 2×SSC and 0.1% Triton X-100 at 37° C. for 15 min each. The colorimetric labels are here fluorescent labels, and according a fluorescence image was obtained with a laser scanner (ScanArray® Lite, Perkin Elmer).

TABLE

| | | | | | |
|---|---|---|---|---|---|
| | | | Start | | |
| Biocept | Gene | Accession | Site | Sequence | 5 mod |

| | | | | | | |
|---|---|---|---|---|---|---|
| 3470 | FRAXA | L29074 | 13705 | gtcaggcgct cagctccgtt t | (SEQ ID NO: 1) | Biotin |
| 3556 | FRAXA | L29074 | 13967 | ctcctccatc ttctcttcag ccctgct | (SEQ ID NO: 2) | Phosphate |
| 3686 | FRAXA | L29074 | 13833 | cggcggcggc ggcggcggcg gcggcggcgg | (SEQ ID NO: 6) | C-6 amino linker |
| 3584 | FRAXA | L29074 | 13893 | cgccgccgcc gccgc | (SEQ ID NO: 3) | CY-3 |
| 3594 | FRAXA | L29074 | 13932 | gctcccggcg ctagcagggc tgaagagaag atg | (SEQ ID NO: 5) | C-6 amino linker |
| 3595 | FRAXA | L29074 | 13964 | catcttctct tcagccctgc tagcgccggg agc | (SEQ ID NO: 4) | CY-3 |

The colorimetric intensity, here the fluorescence intensity (FI), at the FRAXA probe (#3594), which serves as a coamplified internal control, also provides an indication of the efficiency of the PCR. The fluorescence intensities at probes #3586 and #3594 were compared to obtain the desired ratio. The ratio of FI's, i.e, the FI of the STR probe divided by the FI of the internal control probe, was found to be 1.78. Substitution of 1.78 into the derived equation set forth hereinbefore provides the following computation of the number of STRs:
Number of repeats=30+(1.78−1.03)66.4=80
Thus, the result is a calculated value of 80 for the number of the CGG repeats in the unknown sample.

To validate the test, PCR and sequence analysis were used to tediously directly determine the number of CGG repeats; the result was found to be about 80 to 85. The test procedure is thus felt to be fully accurate.

Although the invention has been described with regard to some preferred embodiments which constitute the best mode known at this time to the inventor for carrying out his invention, it should be understood the various changes and modifications may be made without departing from the scope of the invention which is set forth in the claims appended hereto. The disclosures of all U.S. patents and applications and articles referenced are expressly incorporated herein by reference.

Particular features of the invention are emphasized in the claims which follow.

1. A method for detecting a mutation indicative of fragile X syndrome, which method comprises the steps of:
(a) obtaining genomic DNA to be tested,
(b) using PCR to amplify nucleic acid along the X-chromosome in the genomic DNA which includes all of the CGG repeats of the untranslated portion of the FRAXA gene plus a substantial contiguous segment of nucleic acid adjacent to said CGG repeats,
(c) obtaining single-stranded product from the amplified nucleic acid of step (b),
(d) hybridizing colorimetric-labeled oligonucleotides which target for (i) (CGG) repeats and (ii) said contiguous nucleic acid segment with said single-stranded product of step (c),
(e) binding said single-stranded product of step (c) to a solid phase,
(f) separating said hybridized product of step (d) from the remainder of the target material,
(g) recovering the labeled target material from the separated product of step (f),
(h) then hybridizing the recovered labeled target material of step (g) to a microarray having a plurality of spots containing suitable oligonucelotide probes complementary thereto,
(i) following hybridization to the microarray, measuring the calorimetric intensities of the hybridized labeled target material present at specific spots on the microarray to obtain individual values therefor, and
(j) comparing the results of step (i) with results from known control samples to accurately quantify the number of CGG repeats in the FRAXA gene of the obtained genomic DNA.

2. The method of claim 1 wherein the number of CGG repeats is determined using the following formula:
N=30+(A−1.03)66.4 where n is the number of repeats and a is the ratio of the CI of the target which hybridized with CGG probes to the CI of the target which hybridized to the probes for the contiguous nucleic segment.

3. The method of claim 1 wherein said nucleic acid which is amplified includes the CGG repeat section on the X-chromosome and at least a substantial portion of the 3′ translated segment of the FRAXA gene.

4. The method of claim 1 wherein said the hybridizing target for the CGG repeats contains between 3 and 7 triplets and wherein the CGG repeat probes contain between 6 and 20 triplets and at least twice as many triplets as said repeat target.

5. The method of claim 1 wherein the labeled target carries a fluorescent dye at 5′ end thereof.

6. The method of claim 1 wherein step (b) employs pairs of forward and reverse primers which are complementary to the 3′ borders of a DNA region that includes the entire CGG repeat section and said contiguous segment of the nucleic acid containing at least 30 nucleotides.

7. The method of claim 6 wherein said contiguous segment is 3′ of the CGG repeat section and contains at least a substantial portion of the translated segment of the FRAXA gene.

8. The method of claim 6 wherein the forward primer employed in step (b) which is complementary to the 3′ border of the antisense strand of the DNA region has an anchoring moiety at the 5′ end thereof.

9. The method of claim 8 wherein the reverse primer has phosphate at its 5′ end and said single-stranded product obtained in step (c) is obtained by digesting the antisense strand of the double-stranded PCR product with an exonuclease.

10. The method of claim 9 wherein the anchoring moiety is biotin and wherein steps (e) and (f) are carried out following step (d) and separate said hybridized products by binding to avidin that is attached to a solid phase and washing.

11. The method of claim 10 wherein said labeled target material which hybridized in step (d) is recovered in step (g) by treating said hybridized product of step (d) to denature said strands and collecting the supernatant.

12. The method of claim 9 wherein, following step (b), the amplified nucleic acid product is purified to remove unbound primers prior to treating with the exonuclease to obtain the single-stranded target material.

13. The method of claim 6 wherein said forward primer includes SEQ ID NO: 1 and said reverse primer includes SEQ ID NO: 2.

14. A method for detecting a mutation indicative of fragile X syndrome, which method comprises the steps of:
(a) obtaining genomic DNA to be tested,
(b) using PCR and forward and reverse primers to amplify nucleic acid along the X-chromosome in the genomic DNA which includes all of the CGG repeats and a contiguous portion of the translated FRAXA gene, said forward primers having an anchoring moiety at the 5′ end thereof,
(c) purifying the double-stranded product of step(b),
(d) obtaining single-stranded product from step (c) by digesting the antisense strand thereof with an exonuclease,
(e) hybridizing the product of step (d) with fluorescence-labeled antisense targets for (CGG) repeats and for the contiguous portion of the FRAXA gene,
(f) separating said hybridized product of step (d) from the remainder of nonhybridized targets by binding to a solid phase through said anchoring moieties at the 5′ ends of said forward primers,
(g) hybridizing the product of step (g) to a microarray containing suitable probes and, following hybridization to said microarray, measuring the fluorescent intensities of fluorescence-labeled target material present to obtain individual values therefore, and
(h) comparing the results of step (g) with results from known control samples using the following formula:
N=30+(A−1.03)66.4 where N is the number of repeats and A is the ratio of the FI of the target which hybridized with CGG probes to the FI of the target which hybridized to the probes for the contiguous segment, to accurately quantify the number of CGG repeats in the FRAXA gene of the DNA obtained.

15. A method for detecting a short tandem repeat polymorphism (STRP), which method comprises the steps of:
(a) obtaining genomic DNA to be tested,
(b) using PCR to amplify nucleic acid along the chromosome in the genomic DNA which includes all of the STRs of interest plus a substantial contiguous segment of the nucleic acid adjacent to said STRs,
(c) obtaining single-stranded product from the amplified DNA of step (b),
(d) hybridizing colorimetric-labeled oligonucleotides which target for (i) STRs and (ii) said contiguous nucleic acid segment with said single-stranded product of step (c),
(e) binding said single-stranded product of step (c) to a solid phase,
(f) separating said hybridized product of step (d) from the remainder of the labeled target material,
(g) recovering the labeled target material from the product of step (f),
(h) then hybridizing the recovered labeled target material of step (g) to a microarray having a plurality of spots containing suitable oligonucleotide probes complementary thereto,
(i) following hybridization to the microarray, measuring the calorimetric intensities of the hybridized labeled target material present at specific spots on the microarray to obtain individual values therefor, and
(j) comparing the results of step (i) with results from known control samples to accurately quantify the number of STRs in the region of interest of the obtained DNA.

16. The method of claim 15 wherein said the hybridizing target for the STRs contains between 3 and 7 repeats, and wherein the STR probes contain between 6 and 20 repeats and at least twice as many repeats as said STR target.

17. The method of claim 15 wherein step (b) employs a pair of forward and reverse primers which are complementary to the 3′ borders of a DNA region that includes the entire STR section and said contiguous segment of the nucleic acid containing at least 30 nucleotides, the forward primer employed in step (b) being complementary to the 3′ border of the antisense strand of the DNA region and having an anchoring moiety at the 5′ end thereof, and the reverse primer having phosphate at its 5′ end, wherein said labeled target material carry fluorescent dye at 5′ ends thereof, and wherein said single-stranded product is obtained in step (c) by digesting the antisense strand of the double-stranded PCR product with an exonuclease.

18. A kit to detect a mutation indicative of fragile X syndrome, which kit comprises:
(a) a pair of DNA oligonucleotides that will function as forward primers and reverse primers in a polymerase chain reaction (PCR) for amplifying mammalian genomic DNA, wherein the forward primer is complementary to a 3′ nucleotide sequence of the antisense strand of the X-chromosome at a location therealong which is 5′ of the untranslated region of the FRAXA gene and the reverse primer is complementary to a location within the FRAXA gene or 3′ thereof,
said forward primer having an anchoring moiety covalently linked to the 5′ end thereof,
said reverse primer having phosphate at its 5′ end, and
said pair of primer oligonucleotides being specific to amplify the region of genomic DNA which contains all of the CGG repeats and a substantial contiguous segment that serves as an internal control,
(b) labeled oligonucleotides which separately target said CGG repeat region and said internal control segment,
(c) buffers and enzymes for carrying out (i) a PCR, (ii) digestion of the antisense strand, (iii) DNA-DNA hybridizations and washing, (iv) dissociation of hybridized labeled oligonucleotide targets; and (v) colorimetric quantitation,
(d) at least one microarray having a plurality of spots, which spots each have attached thereto DNA probe complementary to one of said labeled oligonucleotide targets; and
(e) means for performing diagnosis for the number of CGG repeats using the results of colorimetric scanning of said microarray and earlier generated data from control samples.

19. The kit of claim 18 wherein one said hybridizing target contains between 3 and 7 CCG repeats, and wherein the CGG probes contain between 6 and 20 repeats and at least twice as many repeats as said complementary target, wherein said forward and reverse primers are complementary to the 3′ borders of a DNA region that includes the entire CGG repeat section and said contiguous segment of the nucleic acid which is 3′ thereof and contains at least 30 nucleotides, the forward primer being complementary to the 3′ border of the antisense strand and having biotin at the 5′ end thereof, and the reverse primer having phosphate at its 5′ end, wherein said labeled target oligonucleotides carry fluorescent dye at 5′ ends thereof, wherein an amount of STR region target at least about 10 times the amount of the internal control region target is provided, and wherein an exonuclease is provided to digest the antisense strand of the double-stranded PCR product to obtain single-stranded PCR product.

20. A kit to detect a mutation indicative of STRP which kit comprises:
(a) a pair of DNA oligonucleotides that will function as forward primers and reverse primers in a polymerase chain reaction (PCR) for amplifying mammalian genomic DNA, said pair of primer oligonucleotides being specific to amplify a selected region of genomic DNA which contains all of the STRs and a substantial contiguous segment that serves as an internal control; wherein the forward primer is complementary to a 3′ nucleotide sequence of the antisense strand of the selected region of the chromosome and the reverse primer is complementary to the 3′ end of the sense strand of the selected region,
said forward primer having an anchoring moiety covalently linked to the 5′ end thereof, and said reverse primer having its 5′ end blocked to elongation,
(b) labeled oligonucleotides which separately target said STR region and said internal control segment,
(c) buffers and enzymes for carrying out (i) a PCR, (ii) digestion of the antisense strand, (iii) DNA-DNA hybridizations and washing, (iv) dissociation of hybridized labeled oligonucleotide targets; and (v) calorimetric quantitation,
(d) at least one microarray having a plurality of spots, which spots each have attached thereto DNA probe complementary to one of said labeled oligonucleotide targets, and
(e) means for performing diagnosis for the number of STRs using the results of calorimetric scanning of said microarray and earlier generated data from control samples.

21. A kit according to claim 20 which includes solid-phase material having coupling agents which are complementary to said anchoring moieties.


SEQUENCE LISTING (6 sequences)

SEQ ID NO: 1 - DNA, 21 bp, Homo sapiens
  misc_feature: Synthetic Primer
gtcaggcgctcagctccgttt

SEQ ID NO: 2 - DNA, 27 bp, Homo sapiens
  misc_feature: Synthetic Primer
ctcctccatcttctcttcagccctgct

SEQ ID NO: 3 - DNA, 15 bp, Homo sapiens
  misc_feature: Synthetic Target Sequence
cgccgccgccgccgc

SEQ ID NO: 4 - DNA, 33 bp, Homo sapiens
  misc_feature: Synthetic Targe sequence
catcttctcttcagccctgctagcgccgggagc

SEQ ID NO: 5 - DNA, 33 bp, Homo sapiens
  misc_feature: Synthetic Probe
gctcccggcgctagcagggctgaagagaagatg

SEQ ID NO: 6 - DNA, 30 bp, Homo sapiens
  misc_feature: Synthetic Probe
cggcggcggcggcggcggcggcggcggcgg